(19) United States
(12) Patent Application Publication
(10) Pub. No.: US 2022/0111032 A1
(43) Pub. Date: Apr. 14, 2022

(54) IMMUNOGENIC COMPOSITION FOR PORCINE EPIDEMIC DIARRHEA VIRUS

(71) Applicants: Chung Yuan Christian University, Taoyuan City (TW); Academia Sinica, Taipei (TW)

(72) Inventors: Tzong-Yuan WU, New Taipei City (TW); Ming-Hao CHANG, Taoyuan City (TW); Hui-Wen CHANG, Taipei City (TW); Yu-Chan CHAO, Taipei (TW); Wei-Ting HSU, Miaoli County (TW)

(73) Assignees: Chung Yuan Christian University, Taoyuan City (TW); Academia Sinica, Taipei (TW)

(21) Appl. No.: 17/065,535

(22) Filed: Oct. 8, 2020

(51) Int. Cl. A61K 39/12; A61K 39/39

(52) U.S. Cl. CPC A61K 39/12; A61K 2039/552; A61K 39/39

(57) ABSTRACT

The present invention is directed to novel immunogenic compositions that protect swine from disease caused by porcine epidemic diarrhea virus (PEDV). The compositions of the invention provide virus-like particles (VLPs) whose effectiveness is enhanced by the selection of preferred adjuvants.

### BACKGROUND OF THE INVENTION

### 1. Field of the Invention

The present disclosure relates to a novel immunogenic composition that protects swine from diseases caused by porcine epidemic diarrhea virus (PEDV).

### 2. Description of Related Art

Porcine epidemic diarrhea (PED) is a contagious enteric viral disease caused by PEDV and is specific for pigs. PEDV contains positive-sense, single-stranded, ˜28-kilobase RNA genome, incorporated with nucleocapsid (N) protein and enveloped in the membranous outer coat comprising three structural proteins: membrane (M), envelop (E), and spike (S) proteins. The S protein is a multifunctional molecular apparatus responsible for specific host and tissue recognition and induction of protective humoral as well as cellular immunities for viral neutralization and elimination. The M protein, the most abundant structural proteins, is responsible for generation of virus particles. The small E protein, accounting for a minority of the envelop component, plays a critical role in the viral morphogenesis and final step of the budding process. PEDV has a tropism for enterocytes of villous tips, and further leads to atrophic enteritis. Pigs at all ages can be affected; however, the severity of the gastrointestinal clinical signs is age-dependent as a result of slower turnover of enterocytes and incomplete innate immunity in neonatal piglets than in postweaning pigs. Historically, PED had a minimal effect on piglets in a sporadic to epidemic manner. However, since 2010, PEDV has evolved into the high-virulent, genogroup 2 (G2) strains from previous G1 strains, accompanied by high mortality in neonatal piglets and devastating global impacts. The high death rates cause tremendous economic losses highlighting the requirement of effective vaccine strategy.

In the past, for the prevention of PEDV infection, the available tools were live-attenuated or inactivated vaccine derived from classical PEDVs, such as strain CV777, DR13, P5-V, or SM98-1. The traditional vaccine failed to confer cross-protection against the novel high-virulent PEDV G2 strains because of the phylogenetic differences among the S proteins of G1 and G2 strains. For virulent G2 virus, two conditionally licensed vaccines were commercialized in the United States using a replication deficient Venezuelan equine encephalitis virus packaging system for expression of PEDV S protein and an inactivated vaccine based on whole virus of non-S INDEL PEDV strain. The third vaccine candidate in commercial development by Vaccine and Infectious Disease Organization-International Vaccine Centre (VIDO-InterVac) is subunit vaccine using mammalian HEK-293 T cell-expressed PEDV S1 proteins. However, the efficacy of all the commercial vaccines to induce solid lactogenic immunity for suckling piglets against the disease was dubious. Many attempts have also been made to develop effective vaccines, but most of them were incapable of inducing mucosal immunity. Although immunogenicity of live attenuated as well as inactivated-based vaccines are considered more effective. However, live-attenuated approaches suffer from genetic reversion via mutation or virulent restoration with wild-type strains in safety concern. Besides, the both approaches are time-consuming in vaccine development. In the quest for novel strategies, the next generation vaccine is qualified to fast deal with high mutation frequency of RNA virus. To ensure the safety of the vaccine, subunit vaccine approach is considered as one of the potential candidates. The subunit strategy possesses the properties of safety while generally losing the optimal immunogenicity. With the improvement of biotechnology, the advanced subunit vaccine—virus-like particle (VLP)—have dealt with itself drawback of the low immunogenicity. VLP is a robust balanced approach maximally avoiding trade-offs between the security and immunogenicity with regard to the vaccine development.

Virus-like particles (VLP) exhibiting the size and geometry of the viral structures, closely resembling the corresponding native virion but without viral genomic nucleic acids, have been suggested to improve the immunogenicity. The absence of the genetic material renders VLP replication-incompetent. Nanometer dimensions in a range of supramolecular particulate antigens (20-200 nm) allow VLP not only to freely drain into lymph nodes but to be efficiently uptaken by antigen-presenting cells (APCs). Uptaken by APCs, VLPs is conducive to T cell responses, including CD4⁺ and CD8⁺ T helper cells. The efficient cross-presentation is mainly contributed by the appropriate size of VLP to activate CD8⁺ lymphoid dendritic cells (DCs), which only reside in lymph nodes. The CD8⁺ DC, in tandem with cytokine secretion (mainly IFN-γ), is essential to cytotoxic immune responses. Apart from cell immunity, VLPs, characterized by nanoparticles with highly repetitive array of conformational epitopes, can readily disseminate into paracortex and directly interact with B cells via both B cell receptors and toll-like receptors (TLR), which facilitate the subsequent activation, proliferation, immunoglobulin class switching, and somatic mutation. Therefore, VLP is an effective and safe tool for potential stimulation of cellular and humoral immunity in the absence of intracellular replication. By contrast, the soluble antigens alone, devoid of pathogen-like size and repetitive organization, have been shown to be poor cross-linking of B cell TLR ligands and lacking induction of MHC class I pathway. When it comes to PEDV VLP compositions, S, M, and E proteins are indispensable. S proteins are a critical effector for stimulation of neutralizing body against PEDV. It has been reported that M and E proteins are necessary for assembling of coronavirus envelope. Interestingly, both severe acute respiratory syndrome (SARS)-coronavirus and Middle East respiratory syndrome (MERS)-coronavirus VLPs were found self-assembled with only S proteins into a smaller nanoparticle (approximately 25 nm) in a quarter diameter of the authentic VLP. Considering the high complexity of the enveloped PEDV VLP, baculovirus expression vector system (BEVS) was adopted due to the unique element, internal ribosome entry sites (IRESs), which allows co-expression of polycistronic genes, and named as polycistronic BEVS, P-BEVS. To ensure each nanoparticle comprises all the compositions, it is necessary to exploit co-expression of the S, M, E proteins in individual cells instead of co-infection in production of the PEDV VLP. A recent report has demonstrated P-BEVS approach can successfully produce PEDV VLP, capable of inducing PEDV-specific humoral immunity in mice. However, the efficacy of the PEDV VLP vaccine against PEDV challenge in pig models is unknown.

The PEDV is categorized as the type I enteropathogenic virus, which could be generally controlled by local humoral and cellular immunity. The establishment of mucosal immunity requires specific microenvironment to promote development of the defense models, such as immunoglobulin class switching to IgA and J chain, and program surface homing ligands or receptors of immune cells. Therefore, in the natural situation, the best immunization route is through the affected compartment. Many studies have proved oral inoculation of virulent enteric viruses had better induction of mucosal IgA. However, oral administration of the equal vaccine dosage is technically difficult and labor-intensive in the swine industry. The operational practicability renders intramuscular injection a common immunization route in the field at the expense of protective mucosal immunity.

Accordingly, there exists in this art, a need of an improved immunogenic composition capable of protecting swine from PEDV infection, particularly from the infection of the high-virulent PEDV G2 strain.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to an immunogenic composition that protects swine from disease caused by porcine epidemic diarrhea virus (PEDV). The immunogenic composition is a virus-like particle (VLP)-based vaccine capable of protecting swine against infection of PEDV. Accordingly, the present immunogenic composition includes at least, a virus-like particle (VLP) that comprises an antigenic PEDV protein; and an adjuvant that comprises a chemokine ligand (CCL). The present immunogenic composition will trigger, upon swine immunization, a strong immune response characterized by the induction of high levels of neutralizing antibodies and interferon-γ secreting cells.

According to preferred embodiments of the present disclosure, the PEDV VLP of the present immunogenic composition comprises three antigenic PEDV proteins, which are the spike (S), membrane (M), and envelope (E) proteins of PEDV.

According to preferred embodiments of the present disclosure, the adjuvant comprises two CCLs, which are CCL25 and CCL28, respectively.

In another aspect, the present disclosure is directed to methods for protecting swine against PEDV infection. The method includes the step of administering to the swine the immunogenic composition of the present disclosure.

According to embodiments of the present disclosure, the PEDV VLP in the immunogenic composition comprises the PEDV S, M, and E proteins.

According to embodiments of the present disclosure, the adjuvant in the immunogenic composition comprises CCL25 and CCL28.

According to embodiments of the present disclosure, the immunogenic composition is orally administered to the swine.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 The organization of the PEDV structure protein sequence and the construction maps of the plasmid pFastBac1-HM6H-PEDV-S-2A-M-Pnv339-eGFP-Rhir-E. The recombinant structural proteins of PEDV were driven by the polyhedrin promoter, and followed by the honey bee melittin signal peptide and 6×His-tag which replaced the original signal peptide from spike. Rest of the spike sequence derived from the Taiwan G2b PEDV-PT strain was going under a codon optimization to insect system and linked with 2A-like sequence and membrane M gene sequence. The envelope protein E was translated through the internal ribosome entry site (IRES) of RhPV. EGFP reporter gene was inserted to the plasmid and expressed by a truncated Perina nuda picorna-like virus IRES (PnV339 IRES). And the resulting recombinant baculoviruses derived from the plasmid pFastBac1-HM6H-PEDV-S-2A-M-Pnv339-eGFP-Rhir-E was named SME-bac.

FIG. 2 The detection of PEDV Spike (S) proteins in the infected Sf21 cells' medium and the VLP production condition determination. Sf21 cells were infected with different condition in T25 flasks, and all the infected Sf21 cells' medium were collected and removed the cell debris centrifugation and filtration. Western blotting analysis of PEDV S protein expressed by SME-Bac was conducted after sucrose cushion and performed with anti-His tag antibodies. The molecular weights of S proteins were approximately above 170 kDa. Positive control: Sf21 cells infected with S-Bac; a recombinant baculoviruses that expressed the PEDV S protein only.

FIG. 3 Detection of the expression of the recombinant spike (S) protein of SME-Bac infected cells. 2×10⁵ Sf21 cells were infected with 5 M.O.I. SME-Bac for 4 days SME-Bac. (B, E, H) The EGFP signal was detected to locate the SME-Bac infection in Sf21 cells. (I) The S protein was detected by a anti-PEDV S conformation-dependent monoclonal antibody, P4B. The positive signals of S protein were detected with an Alexa Flour® 594-conjugated secondary antibody by fluorescent microscope. (C and F) The mock infected cells and SME-Bac infected cells detected without the P4B antibody but with the secondary antibody were used as controls.

FIG. 4 PEDV like particles were generated in SME-Bac recombinant virus infected Sf21 cells' medium. The electron micrographs demonstrated VLPs (black arrowhead) and baculovirus virion (white arrowhead). The sample was prepared from 5 days infection with 1 M.O.I. SME-Bac infected Sf21 cells' medium.

FIG. 5 Systemic PEDV S-specific IgG levels during immunizations. Pigs were immunized with DPBS, VLP, or VLP with CCL25/28 at day 0, 14, and 35. Blood was collected at day 0 (pre-priming), day 14 (2 weeks post priming), day 28, and 49 (2 weeks post twice boosting) for evaluating PEDV specific IgG by the PEDV S¹⁻⁵⁰¹ based ELISA. Data was shown as the average values of the sample-to-positive ratios (S/P ratio) with error bars representing the standard error of the mean (SEM). S/P ratio was defined as the difference between the optical density (OD) values of sample and the negative control and divided by the difference between the OD values of the positive and negative control. [▴] control; [▪] VLP; [●] VLP+CCL25/28. Different alphabetic letters indicated significant differences between different groups (p<0.05).

FIG. 6 Oral PEDV S-specific IgA levels during immunizations. Pigs were immunized with DPBS, VLP, or VLP with CCL25/28 at day 0, 14, and 35. Oral swabs were collected at day 0 (pre-priming), day 14 (2 weeks post priming), day 28, and 49 (2 weeks post the second boosting) for evaluating PEDV specific IgA by the PEDV S¹⁻⁵⁰¹ based ELISA. Data was displayed as the percentage change, which was defined as difference between the treatment and control group, divided by the control group, multiplied by 100. Although levels of the oral IgA in the both treatment groups at day 49 post vaccination appeared similar, post-hoc comparisons of the adjusted data based on the interaction effect of the body weight show significant differences. The error bar represented the SEM of the values of the percentage change at different time-points. [●] VLP; [▪] VLP+CCL25/28. *, p<0.05.

FIG. 7 Titers of plasma neutralizing antibodies against PEDV during immunization. The neutralizing activity against PEDVPT-P5 was performed in Vero C1008 cells. Data was depicted as mean±SEM. [▴] control; [▪] VLP+CCL; [●] VLP.*, p<0.05.

FIG. 8 Levels of PEDV specific Interferon-γ secreting cell count in peripheral blood mononuclear cells (PBMC). The PBMCs were prepared from the peripheral blood of pigs at day 49 post vaccination. Interferon-γ secreting cells were enumerated by the ELISPOT assay. Data was shown as mean±SEM.

FIG. 9 Growth curves for body weight on a week basis. The body weight of pigs in each group was measured every week. The weekly differences in average body weights for each group was expressed as the mean±SEM. [▴] control; [▪] VLP+CCL; [●] VLP.

FIG. 10(A) is a bar diagram depicting the stool consistency scoring of pigs after being challenged with the high-virulent porcine epidemic diarrhea Pintung 52 (PEDV-PT) strain passage 7 in accordance with one embodiment of the present disclosure, in which the Post-challenge pigs were monitored up to 13 days for evaluating stool consistency and fecal viral loads; and the stool was scored by the four types of consistency: 0, normal; 1, loose; 2, semi-fluid; 3 watery;

FIG. 10(B) is a bar diagram depicting the stool consistency scoring of pigs in FIG. 10(A) after being treated with VLP in accordance with one embodiment of the present disclosure;

FIG. 10(C) is a bar diagram depicting the stool consistency scoring of pigs in FIG. 10(A) after being treated with VLP+CCL in accordance with one embodiment of the present disclosure; and

FIG. 10(D) is a line graph depicting the PEDV viral load in feces of pigs in FIGS. 10(A) to 10(C), the viral load was detected by a probe-based quantitative reverse transcription PCR (RT-qPCR) targeting PEDV N gene and was expressed as mean values of log₁₀ RNA copies/ml±SEM. The detection limit of RT-qPCR was 4.7 log₁₀ (copies/ml) marked as dotted line.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

The term “virus-like particle (VLP)” as used herein refers to nonreplicating, viral shell (i.e., devoid of any viral genomic nucleic acid therein). VLPs are generally composed of one or more viral proteins, such as those proteins referred to as capsid, coat, shell, surface and/or envelop proteins, or particle-forming peptides derived from these proteins. VLPs can form spontaneously upon recombinant expression of the proteins in an appropriate expression system. Methods of producing VLPs are known in the art. The presence of VLPs following recombinant expression of viral proteins can be detected using conventional techniques known in the art, such as electron microscopy, immunological characterization as set forth in the working examples of the present disclosure.

The terms “immune response” and “immunological response” are used interchangeably in the present disclosure, and refer to the development in a subject of a humoral and/or a cellular immune response to antigens (e.g., VLPs) present in the present immunogenic composition. A humoral immune response refers to an immune response mediated by antibody molecules, while a cellular immune response is one mediated by T-lymphocytes and/or other white cells. One important aspect of cellular immunity involves an antigen-specific response by cytotoxic T lymphocytes (CTLs). CTLs have specificity for peptide antigens that are presented in association with proteins encoded by the major histocompatibility complex (MHC) and expressed on the surfaces of cells. CTLs may help induce and promote the destruction of intracellular microbes (e.g., PEDV), or the lysis of cells infected with such microbes. Another aspect of cellular immunity involves an antigen-specific response by helper T-cells. Helper T-cells act to help stimulate the function of nonspecific effector cells against cells displaying peptide antigens in association with MHC molecules on their surface. A “cellular immune response” also refers to the production of cytokines, chemokines, and other such molecules produced by activated T-cells and/or other white blood cells, including those derived from CD4+ and CD8+ T-cells. Hence, an immunological response may include one or more of the following effects: the production of antibodies by B-cells; and/or activation of suppressor T-cells, and/or T-cells directed specifically to an antigen or antigens present in the present immunological composition. These responses may serve to neutralize infectivity, and/or mediate antibody complement, or antibody dependent cell cytotoxicity to provide protection to the immunized host (e.g., swine). Such responses can be determined using standard immunoassays and neutralization assays known in the art.

The term “adjuvant” as used herein refers to any material that increases the humoral or cellular immune response to an antigen (e.g., VLPs of the present disclosure). Many factors are taken into consideration in the selection of an adjuvant. An adjuvant should cause a relatively slow rate of release and absorption of the antigens in an efficient manner with minimum toxic, allergenic, irritating, and other desirable effects to the host. To be desirable, an adjuvant should be non-viricidal, biodegradable, capable of consistently creating a high level of immunity, capable of stimulating cross protection, compatible with multiple antigens, efficacious in multiple species, non-toxic and safe for the host (e.g., no injection site reaction). Other desirable characteristics of an adjuvant are that it is capable of micro-dosing, has excellent shelf stability, is easily manufactured, and/or is inexpensive to produce. Further, it is highly desirable to induce either humoral to al or cellular immune response, or both, depending on the requirement of the vaccination scenario. According to preferred embodiments of the present disclosure, the adjuvant suitable for use with antigens in the present immunogenic composition is a chemokine ligand (CCL), particularly a combination of CCL25 and CCL28. Additionally or optionally, well-known conventional adjuvant such as Freund's Complete Adjuvant (FCA); or other materials such as metallic oxides, alum, inorganic chelates of slats, oils, alginates, polysaccharides, caseinates, and the like may be further included in the present immunogenic composition.

The term “effective amount” refers to an amount of the present immunogenic composition effective to produce an immunological response in the recipient. The immunological response may be sufficient for diagnostic purpose or other testing, or may be adequate to prevent signs or symptoms of disease, including adverse health effect or complications thereof, caused by infection of a disease agent (e.g., PEDV). Either humoral immunity or cell-mediated immunity or both may be induced. The immune response of an animal to an immunogenic composition may be evaluated, such as indirectly through measurement of antibody titers, lymphocyte proliferation assays, or directly through monitoring signs and symptoms after challenge with a wild type strain; whereas the protective immunity conferred by the present immunogenic composition can be evaluated by measuring such as reduction in clinical signs (e.g., mortality, morbidity, temperature, overall physical condition, and overall health and performance of the subject). The immune response may comprise, without limitation, induction of cellular and/or humoral immunity as described above.

### 2. The Present Immunogenic Composition

This invention describes the formulation of an immunogenic composition characterized in having a virus-like particle (VLP) that comprises a structure protein of PEDV serving as an antigen, and an adjuvant that enhances immunity response of the VLP thereby confers protection to swine against PEDV infection.

2.1 VLPs

VLPs of the present disclosure may mimic the structure in size, morphology, and biochemical composition of PEDV; however, they are devoid of fully competent viral genome and therefore unable to cause infection in the host (i.e., swine). The lack of viral genome and lack of infectivity of PEDV further eliminate the need of chemical inactivation therefore better preserving the structures, protein conformations and antigenic properties of PEDV proteins expressed on the VLPs, thereby enhance their immunogenicity and potency as vaccines.

Accordingly, each VLPs of the present disclosure carry on its surfaces at least one antigenic PEDV protein selected from the group consisting of spike (S), membrane (M), envelop (E) and a combination thereof of PEDV. These VLPs, in combination with adjuvants (described below), stimulate an immune response that protects swine against PEDV infection. According to some embodiments of the present disclosure, VLPs may be monovalent in that they include only one antigenic PEDV protein displayed on their surfaces. In other embodiments, VLPs may be hetero-oligomeric in that they include more than one kind of antigenic PEDV protein (e.g., 2, 3 or more PEDV proteins) displayed on their surfaces. Preferably, each VLPs comprises 3 antigenic PEDV proteins, which are respectively the spike (S), the membrane (M), and the envelop (E) proteins of the PEDV.

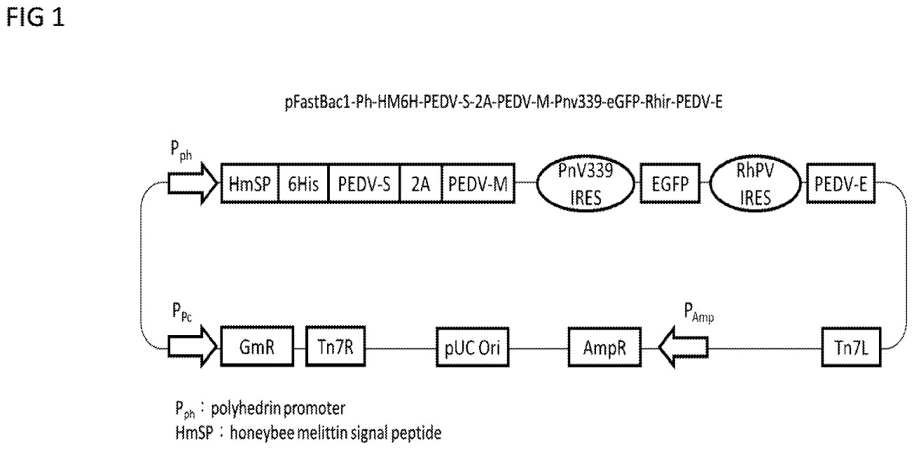

VLPs may be assembled using genetic information comprising segments of the virus genome encoding selected proteins. As depicted in FIG. 1, DNA sequences of three PEDV proteins (i.e., PEDV-S, PEDV-M, and PEDV E proteins) are organized in a single vector capable of expressing simultaneously the transcription units of the three PEDV proteins. The proteins used in the VLPs may be wild-type or modified. Modification include, but are not limited to, substitutions, deletions and/or insertions as well as codon optimization.

The production of VLPs may be achieved by any suitable method, including but not limited to transient and/or stable expression of the protein-encoding sequences in a suspension culture of cells, typically requiring a period of continued cell culture, after which the VLPs are harvested from the culture medium. The VLPs as described herein are conveniently prepared by standard recombinant techniques. Polynucleotides encoding the VLP-forming proteins (e.g., the PEDV-S, PEDV-M and/or PEDV-E proteins) are introduced into a host cell, when the proteins are expressed in the cell, they automatically assemble into VLPs.

Polynucleotide sequence encoding the selected proteins for incorporated into the VLPs can be obtained using recombinant methods, such as by screening cDNA and genomic libraries from cells expressing the gene, or be deriving the gene from a vector known to include the same. For example, plasmids containing sequences that encode naturally occurring or altered cellular products may be obtained from a depository such as American Type Culture Collection (ATCC Manassas, Va., USA) or from commercial sources. Plasmids containing the nucleotide sequences of interest can be digested with appropriate restriction enzymes, and DNA fragments containing the nucleotide sequences can be inserted into a gene transfer vector using standard molecular biology techniques. Alternatively, cDNA sequences may be obtained from cells that express or contain the sequences, using standard techniques, such as extraction and polymerase chain reaction (PCR) of cDNA or genomic DNA. Briefly, mRNA from a cell expressing the gene of interest can be reverse transcribed using oligo-dT or random primers with the aid of reverse transcriptase. The single stranded cDNA may then be amplified by PCR. Alternatively, the nucleotide sequences of interest may also be produced synthetically, rather than cloned, using a DNA synthesizer.

The DNA sequences of interest may be operably linked to each other in any combination. For example, one or more sequences may be expressed from the same promotor and/or from different promoters. Additionally, or optionally, the vector for expressing DNA sequences of interest, which are operably linked to be expressed under the same promoter, may further include a 2A sequence, resulting in the self-cleavage of the expressed proteins. Additionally, or optionally, the vector for expressing DNA sequences of interest may further include an IRES sequence that allows translation in a cap-independent manner. According to preferred embodiments of the present disclosure, the vector for expressing desired antigenic PEDV proteins under same promoter further includes a 2A sequence between the sequences of PEDV-S and PEDV-M, resulting automatic cleavage of the PEDV-S and PEDV-M proteins after expression; and an IRES sequence before the sequences of PEDV-E, thereby achieving cap-independent translation of PEDV-E protein subsequent to the expression of PEDV-S and PEDV-M proteins. By this manner, three antigenic PEDV proteins are simultaneously expressed in a single vector.

Non-limiting examples of vectors suitable for expressing proteins that assemble into VLPs include viral-based expressing vector (e.g., retrovirus, adenovirus, adeno-associated virus, lentivirus), baculovirus vector (e.g., Autographa californica multiple nucleopolyhedrovirus (AcMNPV), Anagrapha falclfera MNPV (AfMNPV), Anticarsia gemmatalis MNPV (AgMNPV), Bombyx mori MNPV (BmMNPV), Buzura suppressaria single nucleopolyhedrovirus (BsSNPV), Helicoverpa armigera SNPV (HaSNPV), Helicoverpa zea SNPV (HzSNPV), Lymantria dispar MNPV (LdMNPV), Orgyia pseudotsugata MNPV (OpMNPV), Spodoptera frupperda MNPV (SfMNPV), Spodoptera exigua MNPV (SeMNPV), and Trichoplusia ni MNPVMNPV), plasmid vector, non-viral vectors, and the like. The expression vectors typically contain coding sequences and expression control elements which allow expression of the coding sequences in a suitable host. Suitable host cells for producing VLPs include, but are not limited to, bacterial, mammalian, baculovirus/insect, yeast cells, and the like.

According to embodiments of the present disclosure, VLPs of the present disclosure are produced in baculovirus/insect cells system. To construct the present recombinant baculoviral vector encoding the PEDV sequences of interest, expression gene cassettes carrying PEDV genes of interest are independently constructed and linked to the promoter sequence (e.g., polyhedron promotor) to produce a transfer vector. The transfer vector is then used with the baculoviral DNA to co-transfect a host cell to produce a recombinant baculovirus, which is capable of propagating in the insect host cell and thereby producing the antigenic proteins of interest respectively encoded by the expression gene cassettes. The recombinant baculovirus was further selected and purified, such as by following the expression of a reporter polypeptide. Suitable insect host cells that may be used in the present disclosure include, but are not limited to, S. furgiperda IPBL-9 (Sf9) cells, Sf21 cells, High Five cells, Minic Sf9 cells and the like. According to some embodiments of the present disclosure, the insect host cells are Sf21 cells. According to embodiments of the present disclosure, the transduction does not significantly affect the viability of the host insect cells.

Depending on the expression system and host cells selected, the VLPs are produced by growing host cells transformed by the expression vectors under conditions whereby particle-forming polypeptides are expressed and VLPs can be formed. The selection of proper growth conditions is within the skill of art. If the VLPs are formed and retained intracellularly, the cells are then disrupted, using chemical, physical or mechanical means, which lysed the cells yet keep the VLPs substantially intact. Alternatively, VLPs may be secreted and harvested from the surrounding culture media. The particles are then isolated (or purified) using methods that preserve the integrity of VLPs, such as by density gradient centrifugation, e.g., sucrose, potassium tartrate, iodixanol gradient centrifugation, and the like; as well as standard purification techniques including e.g., ion exchange and gel filtration chromatography, and the like.

2.2 Adjuvants

VLPs produced as described above can be used directly to elicit an immune response in a subject in the form of a vaccine composition. However, immunity invoked by vaccines that use homogeneous preparations of pathological microorganisms or purified protein subunits as antigens is often poor. The addition of certain exogeneous materials such as an adjuvant therefore becomes necessary. Adjuvant are used to slow the release of antigens from the injection site, and to enhance stimulation of the immune system. The addition of an adjuvant may also permit the use of a smaller dose of antigens to stimulate immune response, thereby reducing the producing cost of the vaccines.

According to embodiments of the present disclosure, VLPs produced as described above, in combination with a chemokine ligand (CCL), when administered to the subject (i.e., swine) will trigger a strong immune response characterized by the induction of high levels of neutralizing antibodies (i.e., IgG and IgA) and interferon-γ secreting cells. There are at least 27 distinctive members of CCL, namely from CCL1 to CCL28, among which CCL10 is the same as CCL9. CCL will induce the migration of monocytes and other cell types such as nature killer (NK) cells and dendritic cells, thus may enhance immunity protection conferred by the present VLPs. Examples of CCL suitable for use with VLPs of this invention include, but are not limited to, CCL25, CCL28 and a combination thereof. Accordingly, the present immunogenic composition comprises VLPs produced as described above, and a combination of CCL25/CCL28. According to embodiments of the present disclosure, equal amounts of CCL25 and CCL28 are mixed with the present VLPs.

Additionally, the immunogenic composition may include other well-known adjuvants in addition to the present combination of CCL25/CCL28. Examples of well-known adjuvants include, but are not limited to, aluminum salts (e.g., aluminum hydroxide, aluminum phosphate, aluminum sulfate), oil-in-water emulsion formulation (e.g., Ribi™ adjuvant system (RAS) (Ribi Immunochem, Hamilton, Mont.)), saponin adjuvant (e.g., Stimulon™ (Cambridge Bioscience, Worcester, Mass.)), Complete Freund's Adjuvant (CFA) and Incomplete Freund's Adjuvant (IFA), and other substances that act as immuno-stimulating agents to enhance the effectiveness of the composition.

2.3 The Present Immunogenic Composition

The present immunogenic composition typically includes an amount of antigens (e.g., VLPs) sufficient to mount an immunological response. An appropriate amount of VLPs may be determined by one of skill in the art. Such amount will fall in a relatively broad range that can be determined though routine trials and will generally be an amount on the order of 0.1 μg to about 10 mg, such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 70, 680, 690, 700, 710, 720, 730, 740, 750, 760, 770, 780, 790, 800, 810, 820, 830, 840, 850, 860, 870, 880, 890, 900, 910, 920, 930, 940, 950, 950, 960, 970, 980, 990, 1,000 μg (or 1 mg), and 2, 3, 4, 5, 6, 7, 8, 9, and 10 mg; more preferably about 0.2 μg to about 5 mg, such as 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 70, 680, 690, 700, 710, 720, 730, 740, 750, 760, 770, 780, 790, 800, 810, 820, 830, 840, 850, 860, 870, 880, 890, 900, 910, 920, 930, 940, 950, 950, 960, 970, 980, 990, 1,000 μg (or 1 mg), and 2, 3, 4, and 5 mg; more preferably about 1.8 mg VLPs.

Additionally, VLPs produced above may be formulated with adjuvants, and/or various additives in appropriate buffers to form the present immunogenic composition. According to embodiments of the present disclosure, VLPs produced above are formulated with equal amounts of CCL25 and CCL28 respectively in the order of 0.1 μg to about 100 such as 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, and 100 μg; preferably, the CCL25 and CCL28 are respectively about 1 μg to about 50 such as 1, 2, 3, 4, 5, 6, 7, 8, 9, 10, 20, 30, 40, and 50 μg; more preferably, the CCL25 and CCL28 are respectively about 30 in appropriate buffers and/or additives, such as carriers, preservatives, antibiotics, stabilizers, immune-modulators, and the like. The present immunological composition can be prepared in freeze-dried (lyophilized) form in order to provide for appropriate storage and maximize the shelf-life of the preparation.

Optionally, a carrier may be present in the immunological composition described herein. Typically, a carrier is a molecule that does not induce production of antibodies harmful to the subject receiving the composition. Suitable carriers are typically large, slowly metabolized macromolecules such as proteins, polysaccharides, polylactic acids, polyglycolic acids, lipid (e.g., liposomes, oil droplets and the like), polymeric amino acids and the like. Examples of suitable immune-modulators for used with the adjuvants described herein include, but are not limited to, IL-1 and IL-2; IL-3 and IL-4; IL-5 and IL-6; IL-7; IL-8, IL-9, IL-10, IL-11, IL-12, and IL-13; IL-14 and IL-15; α-interferon; β-interferon; γ-interferon; tumor necrosis factors (TNFs); WIC class I molecules; WIC class II molecules and the like.

### 3. Use of the Present Immunological Composition

Generally, the present immunogenic composition may be administered to a subject (e.g., swine of any age, whether male or female, irrespective of reproductive status) by any way of delivery, including, for example, by oral, ocular, pulmonary, transdermal, intranasal delivery, or by parenteral injection (e.g., subcutaneously, intraperitoneally, intravenously, intramuscularly, and the like); and may be administered in single dose, or multiple doses within a certain period. Multiple doses may be administered by the same or different routes. The dosage regimen will be determined by the potency of the modality, the vaccine delivery employed, the need of the subject, and etc.

Swine immunized with the present immunogenic composition exhibit a strong immune response characterized by the induction of high levels of neutralizing antibodies and interferon-γ secreting cells. According to embodiments of the present disclosure, secretory IgA, serving as the first line of mucosal immunity, increases significantly after the second boost of the present immunogenic composition (i.e., VLP in combination with CCL25/CCL28). Thus, VLP in combination with chemokines (i.e., CCL25/CCL28) is a promising candidate of mucosal vaccine for other infectious mucosal agents, such as porcine reproductive and respiratory syndrome virus (PRRSV) and human severe respiratory syndrome coronavirus 2 (i.e., SARS-CoV-2)

The following examples are provided to illustrate the present invention without, however, limiting the same thereto.

### EXAMPLES

### Material and Methods

Cells and Viruses

A. albopictus C6/36 and A. aegypti CCL-125 cell clones were maintained in RPMI-1640 medium (Invitrogen) supplemented with 10% fetal bovine serum (FBS), 2 mM L-glutamine, 0.1 mM non-essential amino acids, 1 mM sodium pyruvate, and 2% penicillin-streptomycin at 28° C. in a 5% CO₂ humidified incubator. The Spodoptera frugiperda IPLB-Sf21 (Sf21) cells were grown at 26° C. in TC100 insect medium containing 10% FBS. HEK-293T and Vero-E6 cells were maintained in Dulbecco's modified Eagle's medium (DMEM; Gibco) supplemented with 10% FBS and 2% penicillin-streptomycin at 37° C. in a 5% CO₂ humidified incubator. The Autographa californica nucleopolyhedrovirus (AcMNPV) baculovirus (gene bank accession number NC_001623.1) genome (flashback-ultra) was used to generate recombinant baculoviruses in this study. Viral frozen stocks in respective culture mediums were stored at −80° C.

Plasmid Construction

The S gene, M gene and E gene were derived from the Taiwan G2b PEDV-PT strain (Genbank accession No. KP276252) and S gene was codon optimized to insect system and synthesized by ProTech (ProTech, Taipei, Taiwan). The 2A-like sequence was isolated from Perina nuda virus (PnV) and M gene was cloned into pBac-mcsI-PnV339-eGFP-Rhir-mcsII in XbaI site, and the E gene was then cloned to NotI site on the plasmid. The 2A-M-PnV339-eGFP-Rhir-E sequence and the honeybee melittin signal peptide, 6×His tag contained recombinant S gene were next constructed into pFastBac1 plasmid (Invitrogen, USA) with NEBUILDER® HiFi DNA Assembly kit (NEB) to produce pFastBac1-HM6H-PEDV-S-2A-M-PnV339-eGFP-Rhir-E (FIG. 1) and used it as the recombinant baculovirus transfer vector to recombine with the bacmid DNA in E. coli (strain DH10Bac, Invitrogen). Recombinant bacmid containing the PEDV S gene, M gene and E gene was used to transfect insect cells (Sf21 cell) by Cellfectin (Life Technologies, Carlsbad, Calif., USA) to further generate recombinant baculovirus, SME-Bac.

Generation of VLPs

The Sf21 cells were passaged to a cell density of 1×10⁷ for each T75 flask. After the infection of Sf21 cells with 1 M.O.I. SME-Bac for 5 days, the culture medium was collected, spin in a centrifuge at 600×g for 5 minutes to remove the cell debris, and passed through the 0.22 μm filter. The VLPs in the supernatant was collected through sucrose cushion ultracentrifugation with a condition of 27000 rpm, 4° C. for 90 minutes using the SW-41 rotor. The precipitated VLPs was resuspended with 1×PBS.

Indirect Fluorescent Antibody (IFA) Test

Sf21 cells were passaged to a cell density of 2×10⁵ for each well of 24 well plate. After the infection of Sf21 cells with 5 M.O.I. SME-Bac for 4 days. The cells were washed with 200 μl 1× phosphate-buffered saline with 0.1% tween-20 detergent (PBST) for three times and fixed with 200 μl 4% paraformaldehyde on ice for 20 min. After the removal of fixing buffer, the plate was blocked with 200 μl 3% BSA for 1 hour in room temperature. The previously confirmed anti-PEDV S antibody—P4B (Chang et al., BMC Vet. Res. 2019, 15(1), 421), diluted in blocking buffer (1:200 ratios, 200 μl) was added on the cells at room temperature for 2 hours. After three times PBST wash and 1-hour incubation with Alexa Flour® 594-conjugated AffiniPure Goat anti-Mouse IgG (Jackson ImmunoResearch, Pennsylvania, USA) prepared in blocking buffer with 1:400 ratios, the wells were washed three times with PBST and observed under the fluorescent microscope.

Western Blotting

The precipitated VLPs were loaded to 8% sodium dodecyl sulfate (SDS)-polyacrylamide electrophoresis (PAGE) gel. After electrophoresis, proteins were transferred from gel to a methanol treated PVDF membrane at 300 mA for 180 minutes. The recombinant S protein signals were detected by using rabbit anti-His-tag polyclonal antibody (1:2000 dilution, 600-401-382, Rockland, USA). Then, the goat anti-rabbit IgG conjugated to HRP (1:5000 dilution, 7074S, Cell Signaling Technology, Massachusetts, USA) were used as the secondary antibodies for signal detection. The HRP activity were detected by using the Immobilon™ Western ECL Substrates (Millipore, Mass., USA).

Characterizations of VLPs by Electron Microscopy

For the preparation of the microscopy grids, an aliquot of 10 μL particle samples was added to the carbon-coated grid for 1 min binding and then removed the liquid by 3M filter paper. The grids were later stained with 2% phosphotungstic acid (PTA) for 1 min, the excess PTA was then drained, and the grids were completely dried out 6 hours before examined under transmission electron microscope (FEI Company, Oregon, USA).

Expression and Purification of CC Chemokines

The CC chemokines, CCL25 and CCL28, were prepared in accordance with procedures described previously (Hsueh et al., Vaccines 2020 8(1), 102). The aqueous vaccine formulation comprises immunogen and/or cc chemokines are summarized in Table 1. The amount of VLP and chemokines (i.e., CCL25 or CCL28) were quantified by western blot with ImageJ analysis and bicinchoninic acid assay (Pierce™ BCA protein assay kit, Thermo Fisher scientific, MA, USA).

TABLE 1
Vaccine Formulation of Immunization program

| | | Adjuvant | |
|---|---|---|---|
| Group | Immunogen | CC Chemokine | Freund's Adjuvant |
| Control | None | None | 1^{st}: 0.5 mL |
| | | | Complete Freund's |
| | | | adjuvant |
| VLP | 1.8 mg VLP | None | 2^{nd}: 0.5 mL in- |
| | (0.2 μg Spike | | complete Freund's |
| | protein) | | adjuvant |
| VLP + | 1.8 mg VLP | 30 μg of each | 3^{rd}: 0.5 mL in- |
| CCL25/28 | (0.2 μg Spike | CCL25 and CCL28 | complete Freund's |
| | protein) | | adjuvant |

Cell Lines and Viruses

The high-virulent viral stock of PEDV Pingtung 52 passage 7 (PEDVPT-P7) was derived from the PEDVPT-P5 (GeneBank Accession No. KY929405) as described previously (Kao et al., Viruses 2018 10(10), 543). The Vero C1008 cells (American Type Culture Collection (ATCC) No. CRL-1586) were used for the viral preparation and neutralizing assay, and were maintained in Dulbecco's modified Eagle's medium (DMEM, Gibco, Grand Island, N.Y., USA) supplemented with 10% fetal bovine serum, 250 ng/mL amphotericin B, 100 U/mL Penicillin, and 100 μg/mL Streptomycin. The viral titer of the PEDVPT-P7 was 1.78×10⁵ TCID₅₀/ml, determined by the endpoint titration assay in triplicates using tenfold serial dilution.

Immunization Program of Pigs

Twenty PEDV seronegative and fecal virus-free, three-week-old, male, Large White×Duroc crossbred pigs from the conventional pig farm with no PEDV infection history were selected. These pigs were randomly separated into three groups, including the VLP group (n=7), the VLP+CCL25/28 (n=7), and the control group (n=6). After one-week acclimation, pigs in each group were intramuscularly (IM) primed with 0.5 ml of Dulbecco's phosphate-buffered saline (DPBS, Gibco) or immunogen in DPBS containing 1.8 mg of VLP (0.2 μg spike protein) with or without 30 μg CCL25 and 30 μg CCL28 in combination with 0.5 mL Freund's complete adjuvant (Sigma-Aldrich, St. Louis, Mo., USA) at day 0, followed by twice boosts of the same aqueous formulation admixed with 0.5 ml Freund's incomplete adjuvant (Sigma-Aldrich) at day 14 and day 35. At 0, 14, 28, and 49 days post immunization (DPI), ethylenediamine tetraacetic acid (EDTA) anticoagulated blood and oral and fecal swabs were collected for analyzing the PEDV specific cellular response, systemic IgG, and mucosal IgA titers. At 49 DPI, all pigs were orally challenged with 5 mL of 10⁵ TCID50/ml PEDVPT-P7 to evaluate protective efficacy. Daily monitoring of stool consistency and collecting of fecal swabs for detecting viral shedding were performed. The animal experimental procedure was reviewed and approved by the Institutional Animal Care and Use Committee (IACUC) of the National Taiwan University (Taiwan, Republic of China).

Evaluation of Systemic IgG and Mucosal IgA Levels

PEDV-specific antibodies in plasma and saliva were detected by the in-house PEDV S¹⁻⁵⁰¹-based indirect enzyme-linked immunosorbent assay (ELISA) as established in the previous study (Chang et al., Sci. Rep. 2019 9(1), 2529). The 96-well flat bottom microplates (Thermo Fisher Scientific, Waltham, Mass., USA) were coated with 2 μg/l of the recombinant S¹⁻⁵⁰¹ protein diluted in coating buffer (KPL, Gaithersburg, Md., USA) overnight at 4° C. The S¹⁻⁵⁰¹-coated microplates were washed six times with 200 μL washing buffer (KPL) and then blocked with 300 μL blocking buffer (KPL) for 1 h at room temperature (RT). For evaluation of plasma IgG, 100 μL of 40-fold diluted plasma samples in blocking buffer (KPL) were added per well after washing and kept for 1 h at RT; for salivary IgA, 100 μL of two-fold diluted supernatant of saliva and fecal suspensions in blocking buffer (KPL) were incubated overnight at 4° C. after washing. Following washing as previously described, 100 μL of horseradish peroxidase (HRP)-conjugated goat anti-pig IgG (KPL) at 1:1000 dilution or HRP-conjugated goat anti-pig IgA (Abcam, Cambridge, UK) at 1:5000 dilution was added to detect porcine IgG and IgA, respectively. After 1 h incubation at RT prior to a wash step, 50 μL of ABST® Peroxidase Substrate System (KPL) were introduced for color reaction at RT for 5 min for IgG measurement and 45 min for IgA. The reactions were ended by adding 50 μL stopping solution (KPL). The optical density (OD) values were read at 405 nm by an EMax Plus Microplate Reader (Molecular Devices, Crawley, UK). The IgG titer was expressed as sample to positive ratio (S/P ratio).

Neutralizing Antibody Assay

For evaluation of neutralizing antibody titers, a seeding density of 2×10⁵ Vero cells per mL was placed into 96-well culture plates (Thermo Fisher Scientific, Waltham, Mass., USA), incubated at 37° C., 5% CO₂ overnight to reach nearly full confluency. Plasma samples of pigs were heated at 56° C. for 30 min to inactivate complement. The inactivated plasma samples were 10 time diluted and then 2-fold serial diluted in post-inoculation (PI) medium containing DMEM supplemented with 0.3% tryptose phosphate broth (Sigma, St. Louis, Mo., USA), 0.02% yeast extract (Acumedia, Lansing, CA, USA), and 10 μg/mL trypsin (Gibso). For each well, the mixture of 50 μL of 100 TCID₅₀/mL PEDVPT-P5 and 50 μL of diluted plasma samples was incubated at 37° C., 5% CO₂ for 1 h and were subsequently applied to 90% confluency Vero cells post two-times washing of PI medium for 1 hour of incubation at 37° C., 5% CO₂. After incubation, the mixtures were replaced by the fresh PI medium. The assay was incubated for three days at 37° C., 5% CO₂. Detection of cytopathic effects (CPE) was observed by inverted light microscopy (Nikon, Tokyo. Japan). The neutralizing titer was defined as the last dilution without CPE.

Isolation of Peripheral Blood Mononuclear Cell (PBMC)

For functional assay of PBMC, 9 mL of pig blood was collected in a sterile syringe containing 1 mL of 1% ethylenediamine tetracetic acid (EDTA, Merek, Darmstadt, Germany) at pH 7.5-8.0, and was centrifuged at 3000 rpm for 30 min at 4° C. After centrifugation, the buffy coat was harvested and diluted up to 6 mL with complete Roswell Park Memorial Institute (RPMI) 1640 medium (Gibco, NY, USA) for following density gradient centrifugation. The diluted buffy coat was gently applied to Ficoll-Paque PLUS (GE Healthcare, Uppsala, Sweden) at equal volume of 6 ml, and it was centrifuged at 3000 rpm for 30 min at 20° C. The isolated PBMCs harvested from the cell layer between the RPMI-1640 and Ficoll-Paque PLUS (GE healthcare) were mixed with three times volume of sterile Ammonium Chloride Potassium (ACK) lysis buffer, containing 0.15M NH₄Cl, 1.0M KHCO₃, and 0.01M EDTA at pH 7.2-7.4, for 5 min at 4° C. to remove red blood cells. The erythrocyte-free pellets were washed at 800 rpm for 10 min at 20° C. to get rid of platelets, and then resuspended to a final concentration of 3×10⁶ white blood cells/ml in CTL-Test™ medium (Cellular Technology, LLC, Cleveland, Ohio, USA).

PEDV S¹⁻⁵⁰¹-Specific IFN-γ ELISPOT

According to the manufacturer's instructions, total PEDV spike-specific IFN-γ secreting cells were analyzed by ELISPOT using Anti-porcine IFN-γ pre-coated plates and detection antibodies purchased from Cellular Technology, LLC (Cleveland, Ohio). The 3×10⁵ cells/well of the freshly isolated PBMCs in CTL-Test™ medium (Cellular Technology) was incubated for 24 hours at 37° C. with CTL-Test™ medium (mock) or containing 10 μg/ml of in-house full-length recombinant spike as treatment, or 0.1 μg/ml Concanavalin A (Con A, Sigma-Alderich, MO, USA) as positive control. On the next day, IFN-γ detection and color development were exactly followed the manufacturer's protocol. The scanning and counting of plate and data analysis were performed by Cellular Technology Limited (CTL) team using the CTL IMMUNOSPOT® Analyzer equipped with the IMMUNOSPOT® software version 7.0.23.2.

Stool Consistency Scoring

The severity of diarrhea was categorized using criteria as follows: 0, normal; 1, loose consistency of the stool; 2, semi-fluid consistency of the stool; 3, liquid consistency of the stool (see Hsueh et al., Vaccines 2020 8(1), 102). Body weight of each pig was measured weekly.

RNA Extraction, cDNA Synthesis, and Probed Quantitative Real-Time PCR

To detect fecal viral shedding after the viral challenge, rectal swabs collected every day were resuspended in 900 μL of DPBS (Gibco) and briefly agitated by a vortex. The resuspended samples were centrifuged at 13,000 rpm for 10 min. The supernatant was according to the manufacturer's instructions to automatically extract RNA using the Cador Pathogen 96 QIAcube HT kit (Qiagen, Hilden, Germany). Complementary DNA (cDNA) was reverse-transcribed using the QuantiNova Reverse Transcription Kit (Qiagen, Hilden, Germany) following the manufacturer's protocol as a template for quantitative real time PCR (qPCR). qPCR was perform using our previously published primer-probe set (Jung, K et al., Emerg. Infect. Dis. 2014, 20 (4), 662-665) and under the following thermal conditions: initial denaturation at 95° C. for 2 min, followed by 45 cycles of 95° C. for 15 s, followed by 60° C. for 15 s. The detection limit of the assay was log 4.7 RNA copies per mL based on the standard curve of the in vitro transcribed PEDV RNA.

Statistical Analysis

A p value of 0.05 was adopted for significance. Results were depicted as mean±SEM.

### Example 1 Preparation and Characterization of PEDV VLPs Expressed by Recombinant Baculovirus SME-Bac

1.1 Expression of S Proteins on the Surface of Insect Cells

Recombinant baculovirus SME-Bac was generated by use of the Bac-to-Bac system with pFastBac1-HM6H-PEDV-S-2A-M-Pnv339-eGFP-Rhir-E recombinant baculovirus transfer vector as described in the “Material and Methods” section, the recombinant S protein expression and the VLP production condition were assayed by western blot. The VLP contained supernatant was collected after 3-5 days infection with 1 M.O.I., 3 M.O.I. or 5 M.O.I., respectively.

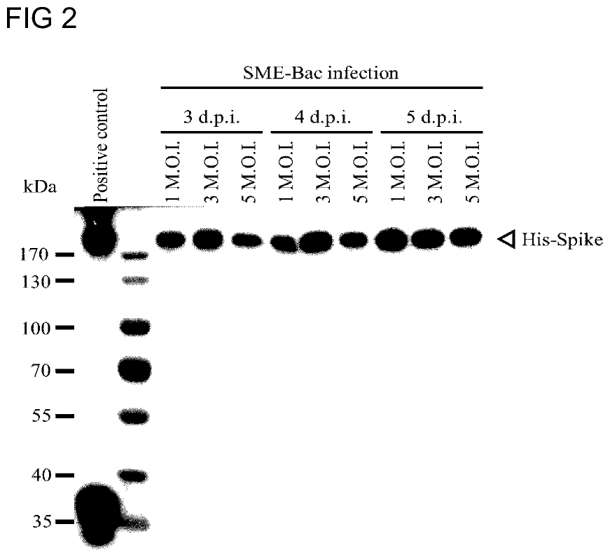

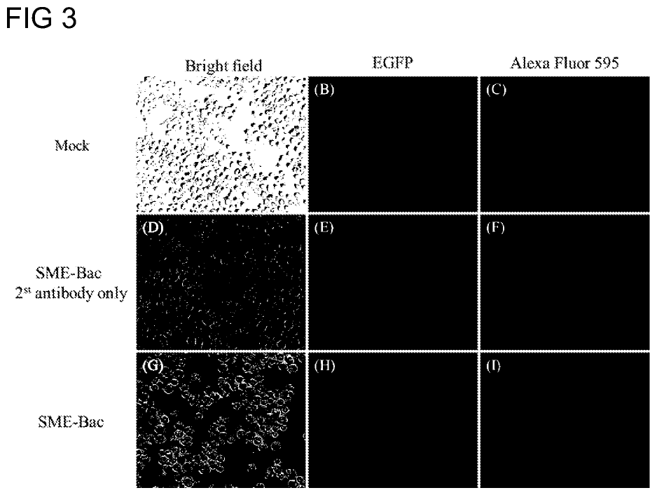

Using the previous characterized PEDV S displayed baculovirus (S-Bac) (Amanna and Slifka, Antiviral Res. 2009, 84(2), 119-130) as a S protein positive control, we confirmed that the His-tagged S protein of PEDV VLPs was present in the culture medium. The positive signals of the S proteins were observed at the size above 170 kDa (FIG. 2), and the optimal VLP production condition would be 5 days infection with 1 M.O.I. To determine if recombinant spike protein had been displayed on VLPs surface, the expressed protein on the surface of the Sf21 cells was detected by an indirect immunofluorescence assay using the PEDV Spike conformation-dependent monoclonal antibody and ALEXA FLOUR® 594-conjugated secondary antibody. It was found that Sf21 cells infected by SME-Bac exhibited a strong fluorescence signal (FIG. 3, (I)), which indicated that the S proteins had been successfully expressed on the surface of SME-Bac infected Sf21 cells.

1.2 Negative Staining Electron Microscopy of PEDV VLPs

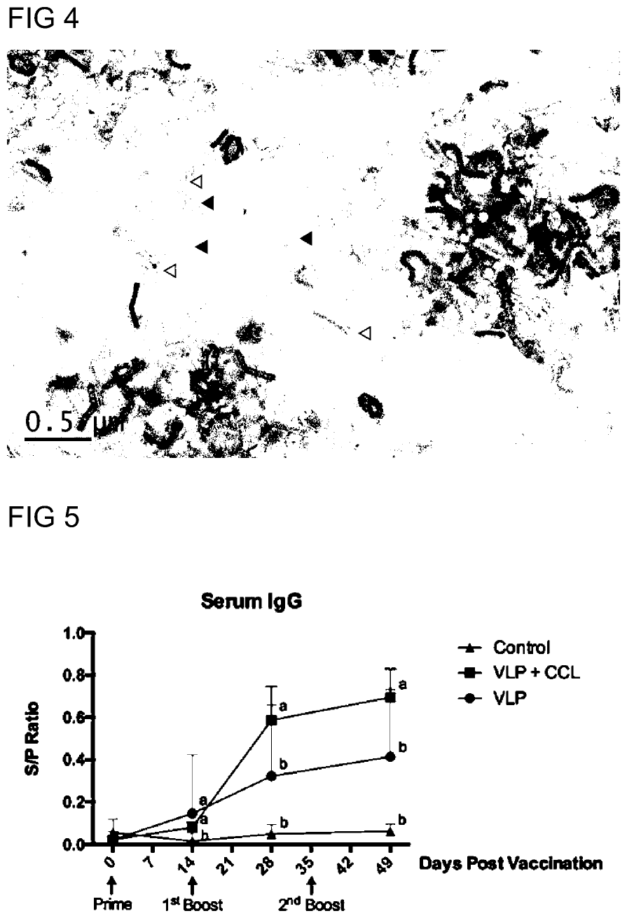

To investigate whether the co-expressed S, M and E protein can be successfully assembled into VLPs, the viral particles from SME-Bac infection were collected and purified from culture supernatant and examined by EM. The EM images as depicted in FIG. 4 revealed numerous VLP particles approximate sizes of 100 nm which are similar to PEDV particles morphology (black arrowhead). There were also some regular long rod-shaped virions with approximate sizes of 200 nm representing the SME-Bac baculovirus virions (white arrowhead).

1.3. Detection of Systemic and Mucosal S-Specific Antibody Titers

As compared with the control group, elevated IgG titer in plasma was observed in both VLP and VLP+CCL immunized groups. While the endpoint titer in the control group was 0.06±0.01, the titer in VLP and VLP+CCL groups after two boosts were 0.41±0.12 and 0.69±0.13, respectively (FIG. 5). Further, significantly higher levels of IgG titers were found for VLP or VLP+CCL groups (as compared to the control) on day 14, and significantly higher level of titers was found for the VLP+CCL group (as compared to those of the control and VLP groups) on day 28 and day 49.

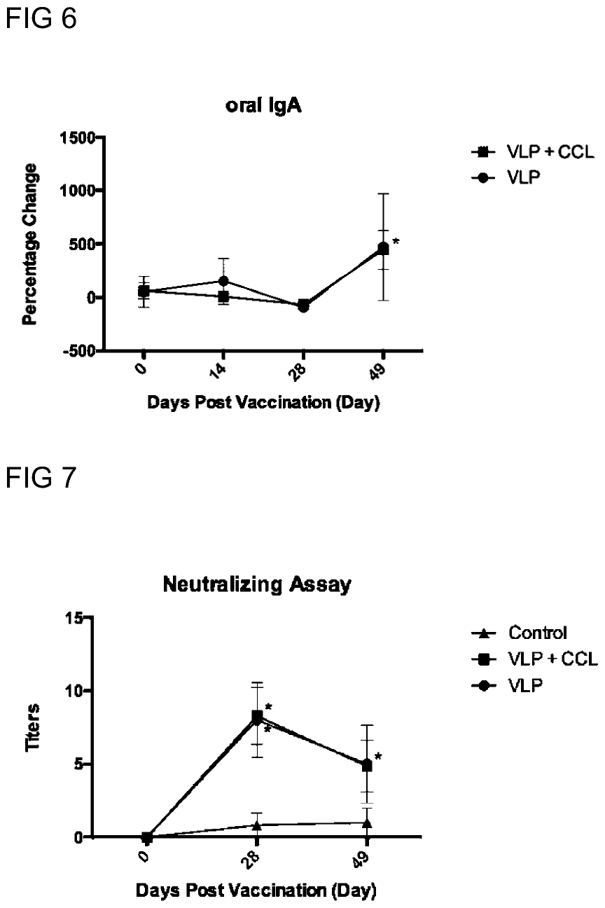

As compared with pigs in the control group, which exhibited the oral IgA S/P titer of 0.03±0.00-0.29±0.08 during the study, increased oral IgA S/P titers of 0.16±0.05 and 0.15±0.05 were observed in VLP and VLP+CCL immunized pigs at two weeks after the second boost, respectively (FIG. 6).

1.4 Evaluation of Neutralizing Antibody Activity in the Blood

To measure the protective efficacy of the generated antibodies, neutralizing assay was used to assess the functional antibodies against homologous viruses. Elevation of the neutralizing antibodies in blood was detected in both VLP and VLP+CCL at 24 DPV but slightly decrease at 49 DPV (FIG. 7).

1.5 Assessment of S-Specific Interferon-γ Secreting Cells in the PBMC

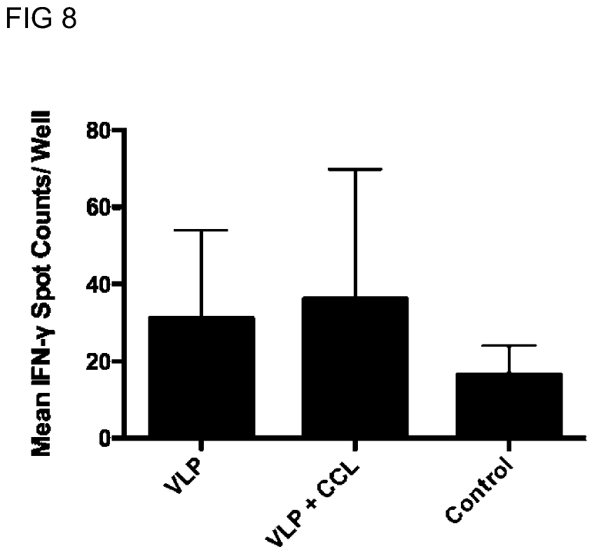

To evaluate the specific cellular immunity against PEDV, we quantified the endpoint IFN-γ secreting T cell in PBMC by ELISPOT assay. Although no significant differences among different groups, the mean values of the VLP and VLP+CCL groups were 31.29±8.59 and 36.14±12.72 spot counts per well and were higher than those in the control group (16.60±7.44 spot counts per well) (FIG. 8).

1.6 Growth Curve of the Body Weight

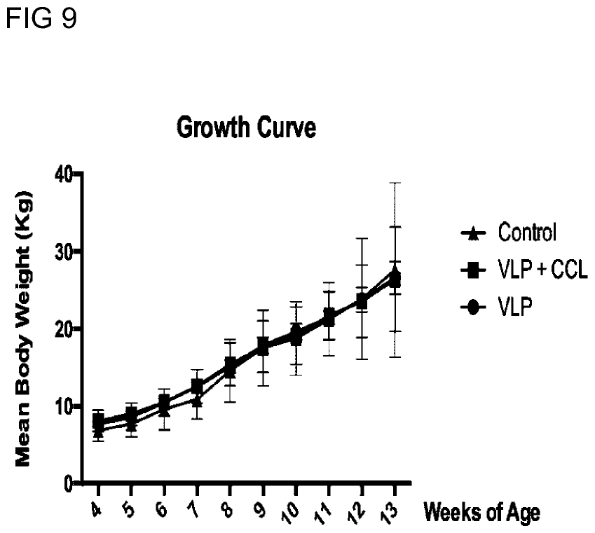

The body weight of each pig was measured on a weekly basis after introduction of the 4-week-old piglet into the experimental facility. The weight gains during the study showed linear growth. The mean body weight demonstrated no significant differences among three groups (FIG. 9).

1.7 Evaluation of Protective Efficacy of VLP Adjuvanted with/without CCL25 and 28 Vaccines Against the Virulent PEDV Challenge

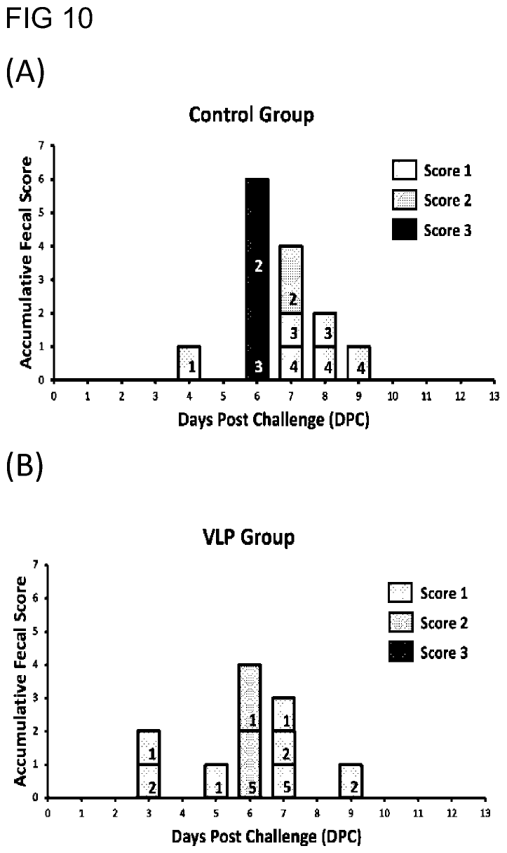

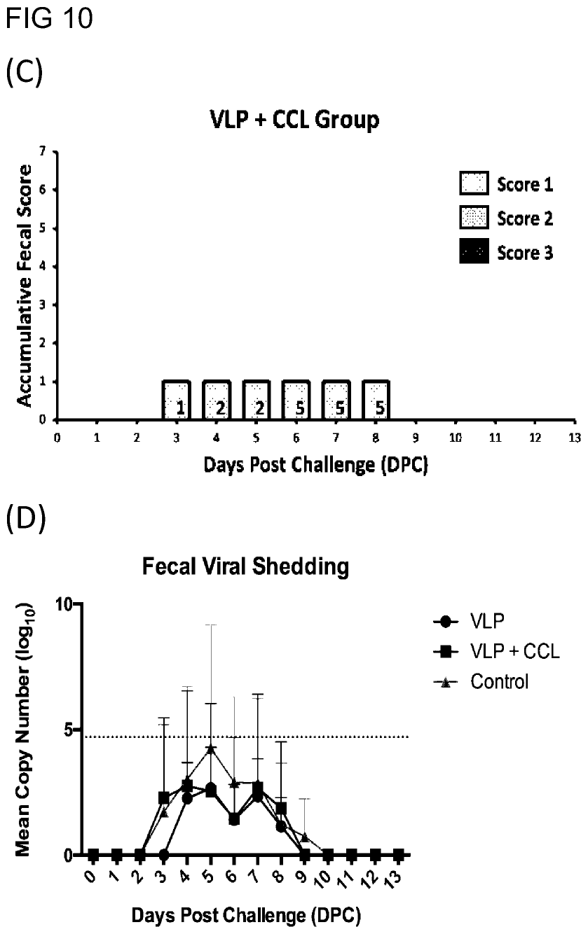

To evaluate the protectivity, all animals were orally challenged with PEDVPT-P7. While one pig showed mild diarrhea (score 1) at 3 DPC, two pigs presented watery diarrhea (score 3) at 4 DPC, none of pigs in the VLP and VLP+CCL groups reached score 3 during the study. All pigs in the control group showed mild to severe diarrhea, followed by gradually decreased total scores during 6-8 DPC, and recovered at 9 DPC. Comparatively, pigs in VLP and VLP+CCL groups showed minimal to moderate diarrhea. While two pigs in VLP group still showed semi-fluid consistency of the stool (score 2) at 6 DPC, all pigs in the VLP+CCL group showed minimal to mild (score 0-1) symptoms. Overall, pigs immunized with VLP and VLP+CCL showed milder diarrhea symptom than those in the control group (FIG. 10, (A) to (C)).

For quantification of PEDV loads in the feces, PEDV shedding in feces was detected by a PEDV N-based real-time RT-PCR. In the control group, viral shedding was detected with a mean value of 1.73±3.46 log₁₀ copies/ml at 3 DPC and reached peak viral shedding of 4.26±4.92 log₁₀ copies/ml at 5 DPC, and declined after 6 DPC. In the VLP-immunized group, viral shedding was detected with a mean value of 2.27±3.18 log₁₀ copies/ml at 4 DPC and fluctuating over 4-8 DPC with peak viral shedding of 2.66±3.65 log₁₀ copies/ml at 5 DPC. Pigs in VLP CCL group exhibited average fecal viral shedding of 2.28±3.20 log₁₀ copies/ml at 3 DPC and lasted for 6 days with peak viral shedding of 2.75±3.79 log₁₀ copies/ml at 4 DPC (FIG. 10(D)).

Taken together, examples of the present disclosure demonstrated that PEDV VLP is capable of eliciting systemic anti-PEDV S-specific IgG and the PEDV S specific IFN-γ producing cells in blood, thereby proving the immunogenicity of the constructed PEDV VLP. Co-administration of CCL25 and CCL28 can modulate immune responses, including enhancement of the systemic anti-PEDV S-specific IgG and induction of mucosal anti-PEDV S-specific IgA. Compared to the control group, the clinical signs of pigs in both VLP and VLP formulated with CCL25/28 groups were markedly reduced with no watery diarrhea and accompanied by lower peak viral shedding. Accordingly, PEDV VLP formulated with CCL25/28 may be a potential PEDV vaccine candidate and the strategy might serve as a platform for other enteric viral vaccine development.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An immunogenic composition comprising,
a virus-like particle (VLP) comprising a protein selected from the group consisting of a spike (S) protein, a membrane (M) protein, an envelope (E) protein and a combination thereof of a porcine epidemic diarrhea virus (PEDV); and
an adjuvant comprising a chemokine ligand (CCL) selected from the group consisting of CCL25, CCL28, and a combination thereof.

2. The immunogenic composition of claim 1, wherein the VLP comprises the proteins of S, M, and E of the PEDV.

3. The immunogenic composition of claim 1, wherein the adjuvant comprises CCL25 and CCL28.

4. A method for protecting a pig against PEDV infection comprising administering to the pig an effective amount of the immunogenic composition of claim 1.

5. The method of claim 4, wherein the VLP of the immunogenic composition comprises the proteins of S, M and E of the PEDV.

6. The method of claim 4, wherein the adjuvant comprises CCL25 and CCL28.

7. The method of claim 4, wherein the immunogenic composition is orally administered to the pig.
